(19) United States
(12) Patent Application Publication
(10) Pub. No.: US 2026/0134541 A1
(43) Pub. Date: May 14, 2026

(54) SYSTEM FOR MACHINE LEARNING AND MRI-BASED EVALUATION OF LUMBAR REGIONS

(71) Applicant: Aclarion, Inc., San Mateo, CA (US)

(72) Inventor: Jeffrey Thramann, Boulder, CO (US)

(21) Appl. No.: 19/429,743

(22) Filed: Dec. 22, 2025

Related U.S. Application Data
(63) Continuation of application No. PCT/US2024/035308, filed on Jun. 24, 2024.
(60) Provisional application No. 63/510,209, filed on Jun. 26, 2023.

(51) Int. Cl. G06T 7/00; A61B 6/00; A61B 6/46; G06V 10/25; G06V 10/764; G06V 10/82; G06V 10/94; G06V 20/50; G06V 20/70

(52) U.S. Cl. CPC G06T 7/0014; A61B 6/463; A61B 6/469; A61B 6/5247; G06V 10/25; G06V 10/764; G06V 10/82; G06V 10/945; G06V 20/50; G06V 20/70; G06T 2200/24; G06T 2207/10024; G06T 2207/10081; G06T 2207/10088; G06T 2207/10108; G06T 2207/20104; G06T 2207/30004; G06V 2201/03

(57) ABSTRACT

A machine learning system is provided to train and use machine learning models to detect lumbar regions of interest on MRI images that correspond to lumbar regions of interest on SPECT/CT images for the same patient and lumbar region.

### CROSS-REFERENCE TO RELATED APPLICATION

The present application is a continuation of PCT Application No. PCT/US2024/035308, filed on Jun. 24, 2024, which claims priority to U.S. Provisional Patent Application No. 63/510,209, filed Jun. 26, 2023, the contents of which are hereby incorporated by reference herein and made part of this specification.

### BACKGROUND

### Field of the Disclosure

This disclosure relates to systems and methods for analysis of internal imaging. More specifically, it relates to novel application of artificial intelligence to magnetic resonance imaging for assessing lumbar regions, including intervertebral discs and facet joints.

### Description of the Related Art

Internal imaging systems, such as magnetic resonance imaging (MRI) machines, single photon emission computed tomography/computed tomography (SPECT/CT) machines, and the like generate imagery of internal body tissue. Health care professionals may use the images to identify regions of interest in the tissue. To aid the health care professionals in identifying regions of interest, various aids such as artificial-intelligence-based image analysis systems may be employed.

### BRIEF DESCRIPTION OF THE DRAWINGS

Embodiments of various inventive features will now be described with reference to the following drawings. Throughout the drawings, reference numbers may be re-used to indicate correspondence between referenced elements. The drawings are provided to illustrate example embodiments described herein and are not intended to limit the scope of the disclosure.

FIG. 1 is a block diagram illustrating an artificial intelligence training system and various internal imaging systems according to some embodiments.

FIG. 2 is a flow diagram of an illustrative routine for training a machine learning model to detect lumbar regions of interest in MRI images based on the presence of lumber regions of interest in corresponding SPECT/CT images.

FIG. 3 is a diagram if an illustrative MRI image of a patient lumbar region and a corresponding SPECT/CT image of the same lumbar region of the same patient according to some embodiments.

FIG. 4 is a block diagram illustrating data flows of an example machine learning model for detecting patient lumbar regions of interest in MRI images according to some embodiments.

FIG. 5 is a diagram illustrating aspects of a MRI procedure, and machine learning model-based analysis of data acquired through the procedure according to some embodiments.

FIG. 6 illustrates various components of an example training system computing device and MRI evaluation system computing device configured to implement aspects of the present disclosure according to some embodiments.

### DETAILED DESCRIPTION

The present disclosure is directed to use of artificial intelligence (AI) to evaluate magnetic resonance imaging (MRI) images to detect lumbar regions of interest. An AI-based lumbar evaluation system can evaluate MRI images using a machine learning (ML) model trained to classify MRI images, or portions thereof, based on patterns learned from single photon emission computed tomography/computed tomography (SPECT/CT) images of lumbar regions. Trained in this way, the ML model can be used to predict which MRI images are likely to represent lumbar regions of interest (ROIs) that would also show up on SPECT/CT images of the same lumbar region for the same patient. For example, conditions such as facet joint arthropathy may present as regions of high color intensity or brightness relative to that of surrounding tissue on a SPECT/CT image, while an MRI image of the same lumbar region of the same patient may not show such obvious indicators of a lumbar ROI. The machine-learned correlation of MRI images to SPECT/CT images can serve as a proxy for detecting or predicting which patients exhibit the conditions that are typically more apparent on SPECT/CT images.

With reference to an illustrative embodiment, a number of MRI procedures and SPECT/CT procedures may be performed on the same patients. The SPECT/CT images may be used as ground truth data for labeling MRI images and training a ML model, such as a convolutional neural network (CNN), region-based CNN (R-CNN), You Only Look Once (YOLO) model, Histogram of Oriented Gradients (HOG), or other model suitable for evaluation of images. Parameters of the ML model may be initialized, and the ML model may be trained in an iterative manner by processing training data images and producing detection output. The detection output may be classification output indicating which regions, if any, of an MRI input image are likely to correspond to lumbar ROIs on corresponding SPECT/CT images of the same patient. The detection output may be evaluated against the ground truth labels for the MRI image to determine the degree to which the detection output differs from the desired output represented. Based on this evaluation for one or more images, the parameters of the ML model may be modified. This process may be repeated in an iterative manner until a desired stopping point is reached. For example, the desired stopping point may correspond to satisfaction of an accuracy metric, exhaustion of a duration of training time or quantity of training iterations, etc.

Various aspects of the disclosure will now be described with regard to certain examples and embodiments, which are intended to illustrate but not limit the disclosure. Although aspects of some embodiments described in the disclosure will focus, for the purpose of illustration, on particular examples of MRI images, SPECT/CT images, AI algorithms, ML models, and training and classification routines, the examples are illustrative only and are not intended to be limiting. In some embodiments, the techniques described herein may be applied to additional or alternative types of MRI images, SPECT/CT images, AI algorithms, ML models, training and classification routines, and the like. In addition, any feature, process, device, or component of any embodiment described and/or illustrated in this specification can be used by itself, or with or instead of any other feature, process, device, or component of any other embodiment described and/or illustrated in this specification.

### Example Execution Environment

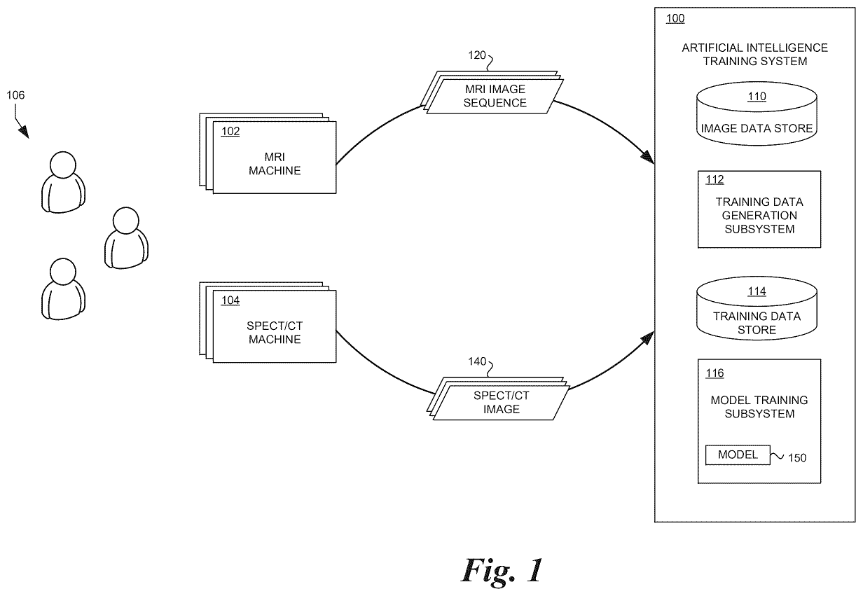

FIG. 1 illustrates example systems and devices for generating training data and training an ML model to detect lumbar ROIs in MRI images. Advantageously, both MRI and SPECT/CT images are obtained for the same patients and used such that the ML model is trained to detect, in MRI images, lumbar ROIs that are typically more apparent (or only apparent) on SPECT/CT images.

In some embodiments, as shown, one or more MRI machines 102 may generate MRI image sequences 120 for each patient 106 in a patient population. For example, the MRI image sequences 120 may include images of the lumbar region of the patients 106. One or more SPECT/CT machines 104 may generate SPECT/CT images 140 for each patient 106 in the same patient population. The SPECT/CT images 140 may be images of the same lumbar regions of the same patients 106 as the MRI image sequences 120. Thus, features present in the SPECT/CT images 140, such as lumbar ROIs, may be used to generate ground truth labels for training a model to detect the features in MRI images.

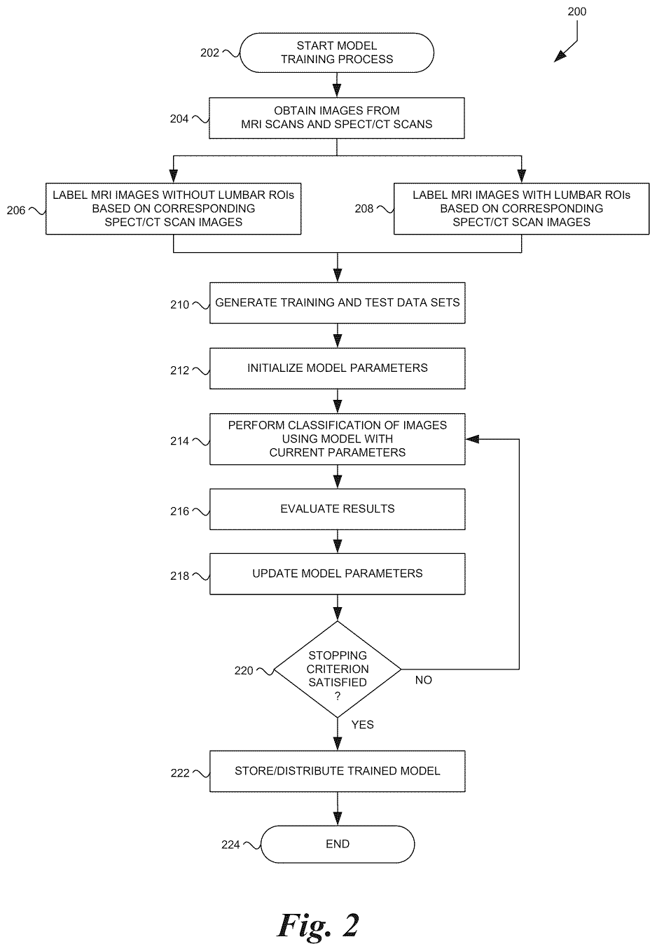

In some embodiments, as shown, an AI training system 100 may include various subsystems and data stores for training an ML model. For example, the AI training system 100 may include an image data store 110 to store MRI image sequences 120 generated by MRI machines 102 and SPECT/CT images 140 generated by SPECT/CT machines 104. The AI training system 100 may also include a training data generation subsystem 112 to generate training data using images from the image data store 110, and a training data store 114 to store the training data. The AI training system 100 may also include a model training subsystem 116 to train a lumbar ROI detection model 150 (also referred to herein simply as model 150 for brevity) using training data from the training data store 114. An example routine that the AI training system 100 may execute to train a model 150 is shown in FIG. 2 and described in greater detail below.

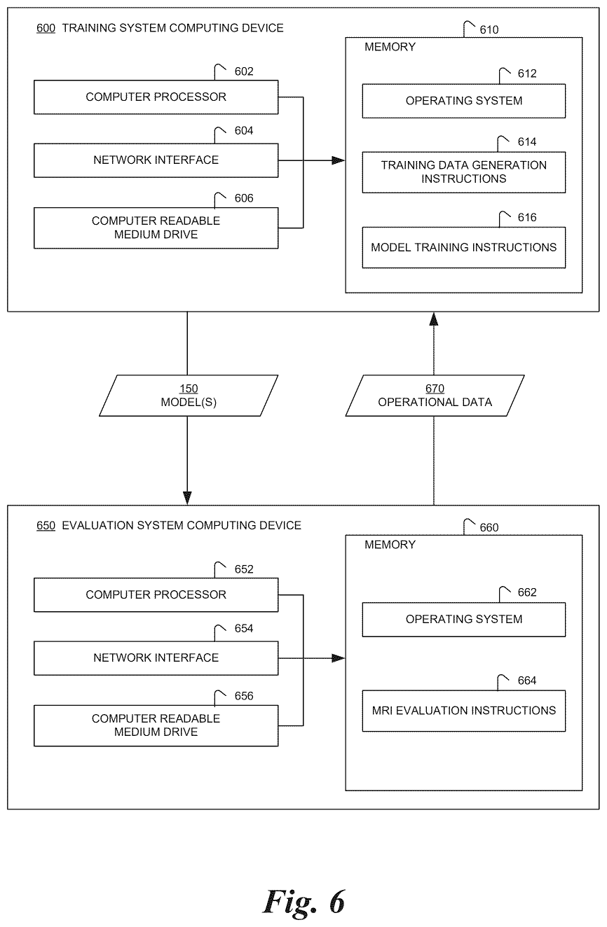

The AI training system 100 may be a logical association of one or more computing systems. The AI training system 100 (or individual components or subsystems thereof) may be implemented on one or more physical computing systems such as blade servers, midrange computing devices, mainframe computers, desktop computers, or any other computing device configured to provide computing services and resources. One example of a training system computing device 600 on which the AI training system 100 or portions thereof may be implemented is shown in FIG. 6. The AI training system 100 may include any number of such computing devices.

In some embodiments, the features and services provided by the AI training system 100 may be implemented as web services consumable via one or more communication networks. In further embodiments, the AI training system 100 (or individual components thereof) are provided by one or more virtual machines implemented in a hosted computing environment. The hosted computing environment may include one or more rapidly provisioned and released computing resources, such as computing devices, networking devices, and/or storage devices. A hosted computing environment may also be referred to as a “cloud” computing environment.

### AI Training for MRI-Based Lumbar Region Evaluation

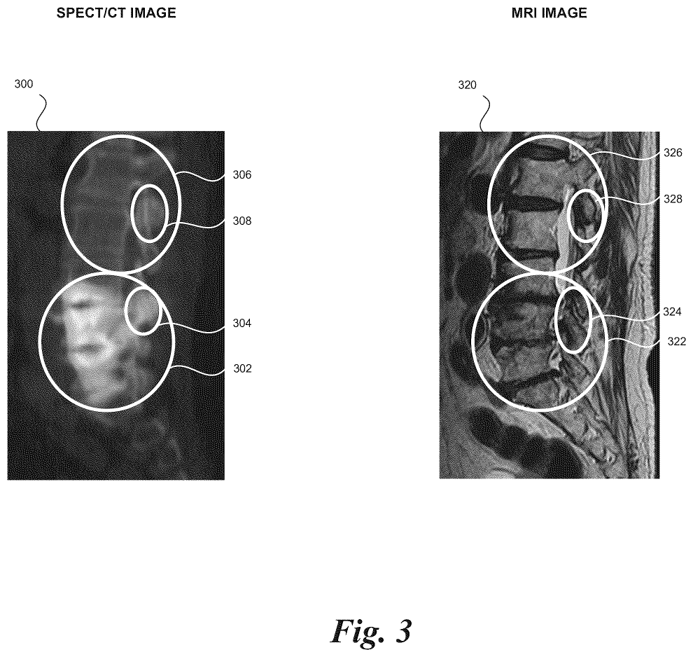

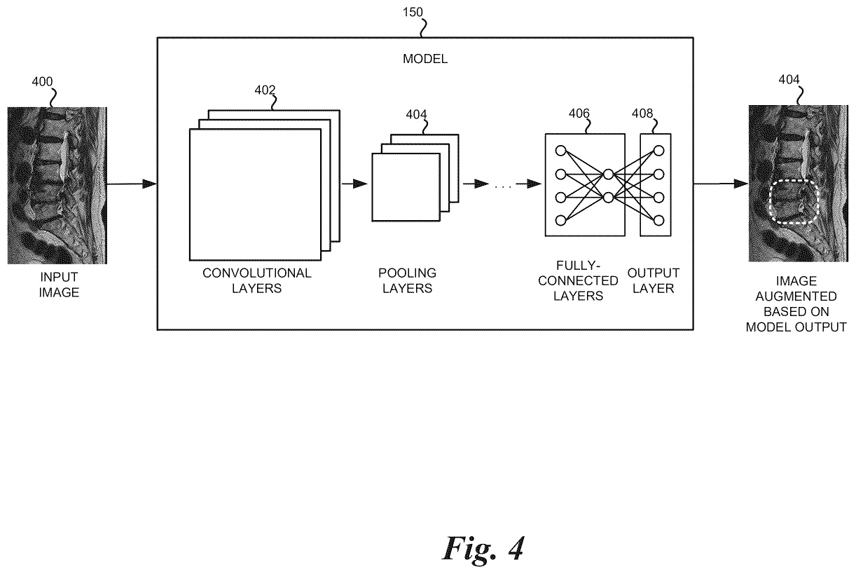

With reference to an illustrative embodiment, FIG. 2 shows an example routine 200 for training a model 150 to detect lumbar ROIs in MRI images. Portions of the routine 200 will be described with further reference to the illustrative SPECT/CT image 300 and MRI image 320 shown in FIG. 3, and the illustrative model 150 shown in FIG. 4.

The routine 200 begins at block 202. The routine 200 may be computer-implemented method that begins in response to an event, such as when the AI training system 100 begins operation, receives a command to train a model, or in response to some other event. When the routine 200 is initiated, a set of executable program instructions stored on one or more non-transitory computer-readable media (e.g., hard drive, flash memory, removable media, etc.) may be loaded into memory (e.g., random access memory or “RAM”) of a computing device, such as the training system computing device 600 shown in FIG. 6 and described in greater detail below. In some embodiments, the routine 200 or portions thereof may be implemented on multiple processors, serially or in parallel.

At block 204, the AI training system 100 (also referred to herein simply as training system 100 for convenience) may obtain MRI image sequences 120 and SPECT/CT images 140 from which to generate training data. The MRI image sequences 120 and SPECT/CT images 140 may be obtained from MRI machines 102 and SPECT/CT machines 104, from an image data store 110 where they were previously stored after receipt from MRI machines 102 and SPECT/CT machines 104, or from some other source. For example, MRI machines 102 and SPECT/CT machines 104 may send images to the training system 100 as the images are generated (e.g., during imaging procedures), after imaging procedures (e.g., in a batch), on demand after a request from the training system 100, on a schedule, or in response to some other event.

In some embodiments, the MRI images 120 or SPECT/CT images 140 may be pre-processed prior to, or as part of the process of, generating training data upon which to train a machine learning model. For example, the resolution of images may be standardized to a resolution upon which the machine learning model is configured to operate (e.g., based on the sizes of various layers of the model 150 described in greater detail below). As another example, images may be segmented into smaller portions for processing instead of, or in addition to, using entire images.

Generally described, an MRI machine 102 uses the magnetization properties of atomic nuclei and the effects of radio frequency (RF) energy to generate MRI image sequences. A magnetic field is employed to align protons within the water nuclei of the tissue being examined. This alignment is disrupted by introduction of external RF energy. The nuclei return to their resting alignment and emit RF energy. The emitted signals are measured, and frequency information may be transformed into intensity levels which are then displayed as pixels in shades of gray. By varying the sequence of RF pulses applied and collected, different types of images are created. Examples of MRI sequence types are T1-weighted and T2-weighted images, with various sequences within each weighing.

A SPECT/CT machine 104 uses the radioactive properties of certain substances, and the varying absorption of the substances into patient tissue to generate SPECT/CT images. Prior to a SPECT/CT scan procedure, a small amount of a radioactive substance is introduced into the patient. The radioactive substance, called a radionuclide or radiotracer, tends to collect within tissue at spots of abnormal physical or chemical change. The radiotracer emits gamma radiation that is detected by the SPECT/CT machine 104 and processed into images. The areas of greater uptake of radiotracer—or otherwise areas where the radiotracer collects—may be referred to as “hot spots,” and may indicate the presence of conditions such as arthritis, tumors, infections, trauma, or other conditions. Hot spots appear on SPECT/CT images as areas with different colors or degrees of brightness in comparison with areas of surrounding tissue.

In certain cases, hot spots on SPECT/CT images may be used to identify conditions that are not as readily apparent in an MRI image. FIG. 3 illustrates an example SPECT/CT image 300 of the lumbar region of a particular patient. The SPECT/CT image 300 shows a hotspot 302 at the location of the intervertebral discs at L3/4 and L4/5. The hotspot 302 also encompasses a facet joint at region 304. The hotspot 302 appears as a region of significantly different color and brightness compared with surrounding tissue, including adjacent portions of the spine. For example, region 306 which includes intervertebral discs at L1/2, L2/3, and L3/4 and a facet joint at region 308, appears with less color change and less brightness in comparison with the surrounding tissue than does hotspot 302. In this example, hotspot 302 may by indicative of a condition, such as facet joint arthropathy of the facet joint in region 304. Thus, hotspot 302 as a whole, or region 304 in particular, may be tagged as a lumbar ROI.

FIG. 3 also includes an example MRI image 320 of the same lumbar region of the same patient as the SPECT/CT image 300. Region 322, which includes the L3/4 and L4/5 intervertebral discs and a facet joint at region 324, corresponds to the same anatomical region as hotspot 302 in SPECT/CT image 300. Region 326, which includes intervertebral discs at L1/2, L2/3, and L3/4 and a facet joint at region 328, corresponds to the same anatomical region as region 306 in SPECT/CT image 300. Although differences in shading of region 322 in comparison with region 326 are visible, the differences are not as apparent as the change in color and brightness that distinguish hotspot 302 from surrounding tissue in the SPECT/CT image 300. In some cases, less distinctive and less apparent shading than shown in FIG. 3 may be present in an MRI image, while a corresponding hotspot may still be distinctive and apparent in a SPECT/CT image. Moreover, the differences in shading of region 322 in comparison with region 326 may not necessarily be indicative of the same conditions as associated with the cause of hotspot 302 in SPECT/CT image 300.

Advantageously, by identifying the regions within MRI images that correspond to lumbar ROIs (e.g., hotspots) in SPECT/CT images of the same patient, a machine learning algorithm can be executed to learn to identify features within the MRI images that are indicative of lumbar ROIs that present in corresponding SPECT/CT images. In addition, the machine learning algorithm can learn to distinguish MRI images with features indicative of such lumbar ROIs from MRI images that do not have features indicative of lumbar ROIs, such as MRI images for which a corresponding SPECT/CT image does not include a lumbar ROI. To execute such a machine learning algorithm and train a lumbar ROI detection model 150, the training data generation subsystem 112 may use SPECT/CT images to label corresponding MRI images as including or not including lumbar ROIs.

Returning to routine 200, at block 206 the training data generation subsystem 112 may use SPECT/CT images 140 to label corresponding MRI images 120 that do not include lumbar ROIs to be detected by the model 150. In the description that follows, a “corresponding MRI image” is an image of the same lumbar region of the same patient captured within substantially the same timeframe (e.g., pre-treatment or pre-diagnosis) as a SPECT/CT image being discussed. Similarly, a “corresponding SPECT/CT image” is an image of the same lumbar region of the same patient captured within substantially the same timeframe (e.g., pre-treatment or pre-diagnosis) as an MRI image being discussed.

In some embodiments, a portion of the SPECT/CT images 140 may have been previously tagged as being negative for the presence of a lumbar ROI. For example, during or after the process of generating images, a health care professional (HCP) or other user may indicate SPECT/CT images 140 that are negative for the presence of a lumbar ROI. Tag data may be incorporated into the SPECT/CT images 140 or corresponding MRI images 120, or provided to the training system 100 as metadata separately from the images. The tag data may include a flag or other indicator of whether there is no lumbar ROI in the corresponding image. The training data generation subsystem 112 may access the tag data and, based thereon, label a portion of the MRI images 120 as not including a lumbar ROI. The labeled images may be stored as training data in the training data store 114.

In some embodiments, a portion of the SPECT/CT images 140 may not have been previously tagged as being negative for the presence of a lumbar ROI. For such images, the training data generation subsystem 112 may generate or otherwise obtain labels for the corresponding MRI images 120 that are negative for the presence of a lumbar ROI. For example, the training data generation subsystem 112 may provide a user interface for HCPs or other users. The user interface may be a graphical user interface delivered as a web page, mobile application interface, desktop application interface, or via some other mechanism of delivery. Users may use the interface to view SPECT/CT images 140 and indicate one or more of: which images do and/or do not include lumbar ROIs; where any lumbar ROIs are located within individual images; more detailed information regarding the lumbar ROIs, etc. Interactions to indicate the presence or absence of lumbar ROIS (or other associated information) can be used to generate tag data that may be incorporated into the SPECT/CT images 140 or the corresponding MRI images 120, or provided to the training system 100 as metadata separately from the images. The tag data may include a flag or other indicator of whether there is no region of interest in the corresponding MRI image. The training data generation subsystem 112 may access the tag data and, based thereon, label a portion of the MRI images 120 as not including a lumbar region of interest. The labeled images may be stored as training data in the training data store 114.

At block 208, the training data generation subsystem 112 may label a subset of the MRI images 120 that include a lumbar ROI to be detected by the model 150. In some embodiments, a portion of the SPECT/CT images 140 may have been previously tagged as being positive for the presence of a lumbar ROI. For example, during or after the process of generating images, an HCP or other user of a SPECT/CT machine 104 may indicate images that are positive for the presence of a lumbar ROI. Tag data may be incorporated into such images—or provided to the training system 100 as metadata separately from the images. The tag data may include a flag or other indicator of whether there is any region or regions of interest in the corresponding image, where in the image the ROI(s) may be located, additional information regarding the nature of the ROI(s) (e.g., whether they are indicative of a patient experiencing facet joint arthropathy), etc. Illustratively, the tag data may indicate a coordinate location of an ROI, an offset of an ROI from a reference location (e.g., center, corner, or edge of an image), a range of locations for a region or regions of interest, or some other data from which the training data generation subsystem 112 can determine the location, size, and/or nature of the ROI(s) and label corresponding MRI image(s) accordingly. The training data generation subsystem 112 may access the tag data and, based thereon, label a portion of the corresponding MRI images 120 as including a lumbar ROI, and in some cases where the ROIs are in each such image. Illustratively, labeling of an image to indicate a lumbar ROI may include generating labeling data from the tag data, or copying the tag data, to indicate a coordinate location of an ROI, an offset from a reference location of an ROI, a range of locations for a region or regions of interest, or some other data from which the training system 100 can train the model 150 to detect the location, size, and/or nature of the ROI(s) in an MRI image 120. The labeled images may be stored as training data images in the training data store 114.

In some embodiments, a portion of the SPECT/CT images 140 may not have been previously tagged as being positive for the presence of a lumbar ROI. For such images, the training data generation subsystem 112 may generate or otherwise obtain labels for those SPECT/CT images 140 that are positive for the presence of a lumbar ROI. For example, as described above with respect to images that are negative for the presence of a lumbar ROI, the training data generation subsystem 112 may provide a user interface for HCPs or other users to view SPECT/CT images 140 and indicate one or more of: which images do and/or do not include lumbar ROIs; where any ROIs are located within individual images; more detailed information regarding the ROIs, etc. Interactions to indicate the presence or absence of lumbar ROIs (or other associated information) can be used to generate tag data that may include a flag or other indicator of whether there is a ROI in the SPECT/CT image 140, the size of the region, the nature of the region, etc. The training data generation subsystem 112 may access the tag data and, based thereon, label a portion of the corresponding MRI images 120 as including a lumbar ROI, the size of the ROI, the nature of the ROI, etc. The labeled MRI images 120 may be stored as training data images in the training data store 114.

Although blocks 206 and 208 are shown as separate blocks in parallel paths of execution, the illustration is an example only and is not intended to limiting. In some embodiments, operations associated with blocks 206 and 208 may be performed serially, with one block occurring before the other. In some embodiments, the operations associated with blocks 206 and 208 may be performed in one step, during which images are analyzed, some images are labeled as negative for regions of interest, and others are labeled as positive for a region of interest, without regard to the order in which the respective images are processed.

At block 210, the training data generation subsystem 112 or some other subsystem of the training system 100 may select training data to be used during the current instance of the routine 200 to train the model 150. In some embodiments, the training data generation subsystem 112 may separate the labeled training images in the training data store 114 into a training set and a testing set. The training set may be used as described in greater detail below to train the model 150. The testing set may be used to test the trained model 150. Advantageously, using a separate testing set of images to test the performance of the model 150 can help to determine whether the trained model 150 can generalize the training to new images that were not presented to the machine learning model during training (or during an iteration of training).

At block 212, the model training subsystem 116 can initialize the parameters of the model 150 to be trained. In some embodiments, the machine learning model may be implemented as a neural network (NN).

Generally described, NNs-including CNNs, deep neural networks (DNNs), recurrent neural networks (RNNs), other NNs, and combinations thereof—have multiple layers of nodes, also referred to as “neurons.” Illustratively, a NN may include an input layer, an output layer, and any number of intermediate, internal, or “hidden” layers between the input and output layers. The individual layers may include any number of separate nodes. Nodes of adjacent layers may be logically connected to each other, and each logical connection between the various nodes of adjacent layers may be associated with a respective weight. Conceptually, a node may be thought of as a computational unit that computes an output value as a function of a plurality of different input values. Nodes may be considered to be “connected” when the input values to the function associated with a current node include the output of functions associated with nodes in a previous layer, multiplied by weights associated with the individual “connections” between the current node and the nodes in the previous layer. When a NN is used to process input data in the form of an input vector or a matrix of input vectors (e.g., data representing an image, such as the values of the individual pixels of the image), the NN may perform a “forward pass” to generate an output vector or a matrix of output vectors, respectively. The input vectors may each include n separate data elements or “dimensions,” corresponding to the n nodes of the NN input layer (where nis some positive integer, such as the total number of pixels in an input image). Each data element may be a value, such as a floating-point number or integer (e.g., a greyscale value or a red-blue-green or “RBG” value of a pixel). A forward pass typically includes multiplying input vectors by a matrix representing the weights associated with connections between the nodes of the input layer and nodes of the next layer, applying a bias term, and applying an activation function to the results. The process is then repeated for each subsequent NN layer. Some NNs have hundreds of thousands or millions of nodes, and millions of weights for connections between the nodes of all of the adjacent layers.

The trainable parameters of the NN include the weights (and in some embodiments the bias terms) for each layer that are applied during a forward pass. In some embodiments, to initialize the parameters of the machine learning model, the model training subsystem 116 can use a pseudo-random number generator to assign pseudo-random values to the parameters. In some embodiments, the parameters may be initialized using other methods. For example, a model 150 that was previously trained using the routine 200 or some other process may serve as the starting point for the current iteration of the routine 200.

At block 214, the model training subsystem 116 can analyze training data images using the model 150 to produce training data output. Illustratively, the training data output may correspond to classification determinations regarding whether training data images are negative or positive for lumbar ROIs, which portions of the images are likely to be negative or positive for lumbar ROIs, or the nature of the ROIs. In subsequent blocks of the routine 200, the training data output is used to evaluate the performance of the model 150 and apply updates to the trainable parameters.

With reference to FIG. 4, the structure and operation of illustrative embodiment of a model 150 to generate training data output (and, similarly, prediction output in production implementations of the trained model 150) will be described. The illustrative model 150 is implemented as a CNN. As shown, the model 150 includes one or more convolutional layers 402, one or more max pooling layers 404, and a set of fully-connected layers 406 before an output layer 408. The convolutional layers 402 and max pooling layers 404 are used to iteratively “convolve” (e.g., use a sliding window to process portions of) an input image 400 and determine a degree to which a particular “feature” (e.g., an edge or other aspect of an object to be detected) is present in different portions of the input image 400. Aspects of this procedure may also be referred to as “feature mapping.” The procedure may be performed using any number of sets of convolutional layers 402 and max pooling layers 404 (e.g., 1, 2, 5, 10, or more sets). The result that is generated by the sets of convolutional layers 402 and max pooling layers 404 may be a matrix of numbers, such as floating-point numbers. The matrix may then be converted to a vector for processing by the set of fully-connected layers 406. The fully-connected layers 406 can generate classification output indicating whether the input image 400 is positive or negative for a lumbar ROI. For example, a particular output value or set of output values may represent a classification as positive or negative (e.g., a value>=0.5 indicates a positive classification, a value<0.5 indicates a negative classification). In some embodiments, the output of the fully-connected layers 406, or separate output generated by or otherwise derived from output generated by the convolutional layers 402 and max pooling layers 404, can indicate the location(s) within the input image 400 that include a region of interest, the nature of a region of interest, etc.

An example of the processing performed by the model 150 will now be described with reference first to the operation of the fully-connected layers 406 at the end of the model 150 and then to the convolutional and max pooling layers 402 and 404 at the beginning of the model 150. The set of fully-connected layers 406 may include an input layer by which output of the convolutional layer(s) 402 and max pooling layer(s) 404 is received. The set of fully-connected layers 406 includes the input layer with a plurality of nodes, one or more internal layers each with a plurality of nodes, and an output layer with a plurality of nodes. The specific number of layers shown in FIG. 4 is illustrative only, and is not intended to be limiting. In some models 150, the set of fully-connected layers 406 may include different numbers of internal layers and/or different numbers of nodes in the input, internal, and/or output layers.

The connections between individual nodes of adjacent layers of the set of fully-connected layers 406 are each associated with a trainable parameter, such as a weight and/or bias term, that is applied to the value passed from the prior layer node to the activation function of the subsequent layer node. For example, the weights associated with the connections from the input layer to an internal layer to which it is connected may be arranged in a weight matrix.

Illustratively, a vector representing output of the convolutional layer(s) 402 and max pooling layer(s) 404 may be computed or otherwise obtained by a computer processor that stores or otherwise has access to the weight matrix. The processor then multiplies the vector by the weight matrix to produce an intermediary vector. The processor may adjust individual values in the intermediary vector using an offset or bias that is associated with the internal layer (e.g., by adding or subtracting a value separate from the weight that is applied). In addition, the processor may apply an activation function to the individual values in the intermediary vector.

The output layer of the model 150 makes output determinations from the last intermediary vector. Weights associated with the connections from the last internal layer to the output layer may be arranged in a weight matrix used to produce an output vector using the process described above with respect to the input layer and first internal layer. The output vector may include data representing the classification or regression determinations made by the model 150 for the input image 400. Some models 150 are configured make u classification determinations corresponding to u different classifications (where u is a number corresponding to the number of nodes in the output layer). The data in each of the u different dimensions of the output vector may be a confidence score indicating the probability that the input image 400 is properly classified in a corresponding classification. Some models 150 are configured to generate values based on regression determinations rather than classification determinations, or regression determinations that correspond to classification determinations.

The training data from which the images 400 are drawn may also include reference data output vectors. Each reference data output vector may correspond to a training image 400, and may include the “correct” or otherwise desired output that the model 150 should produce for the corresponding training image 400. For example, a reference data output vector may include scores indicating the proper classification(s) for the corresponding training image 400 (e.g., scores of 1.0 for the proper classification(s), and scores of 0.0 for improper classification(s)). As another example, a reference data output vector may include scores indicating the proper regression output(s) for the corresponding training data input vector. The goal of training may be to minimize the difference between the output vectors and corresponding reference data output vectors.

Prior to the set of fully-connected layers 406, the image 400 may be analyzed using one or more convolutional layers 402 and one or more max pooling layers 404. Like the set of fully-connected layers 406, the convolutional layers 402 are associated with trainable parameters (e.g., weights, biases) that are applied to portions of layer input, such as portions of the image 400, portions of a prior convolutional layer 402 output, or portions of a max pooling layer 404 output. However, unlike the fully-connected layers 406, the nodes in a convolutional layer 402 may only be connected to a small region of the preceding layer instead of all of the neurons in a fully-connected manner.

By way of illustration, a training image 400 may be represented as a matrix (e.g., for a greyscale image) or a tensor (e.g., for an RGB image with three color channels) of values in which individual values represent individual pixel values of the image 400. A convolutional layer 402 can generate layer output for nodes connected to particular regions in the input image 400. For example, each node of a convolutional layer 402 corresponds to a dot product of its associated weights and a region of the prior layer (or input image 400). There may be more than one feature for which input is being assessed for detection, and the existence of each feature may be assessed using a separate “filter” represented by a set of weights. Thus, in some embodiments the output of a given convolutional layer 402 may be represented as three-dimensional tensor with two dimensions corresponding to spatial dimensions of the input image 400 and a third dimension corresponding to the number of filters. An activation function may also be applied elementwise to each node. These operations may be performed substantially as described above with respect to general NNs and the set of fully-connected layers 406, with adjustment for the limited connectivity of the convolutional layer. A max pooling layer 404 may effectively perform a compression operation on the output of a preceding convolutional layer 402 resulting in max pooling layer output that is reduced in spatial dimensions with respect to the size of the input image 400.

A model 150 implemented as shown and described above thus transforms an input image 400 from the image's pixel values to the final detection scores (e.g., classification or regression scores) output by the model 150. In doing so, the convolutional layers 402 and fully-connected layers 406 perform transformations that are a function of not only their respective inputs (e.g., the inputs from prior layers), but also of the parameters of the layers (the weights and biases of the neurons). Other portions of the model 150 may not have separate trainable parameters. For example, the max pooling layers 404 and any activation functions may implement fixed functions that depend only on their respective inputs and are not necessarily trainable.

Returning to the routine 200 shown in FIG. 2, at block 216 the model training subsystem 116 can evaluate the results of processing one or more training input images 400 using the model 150. In some embodiments, the model training subsystem 116 may evaluate the results using a loss function, such as a binary cross entropy loss function, a weighted cross entropy loss function, a squared error loss function, a softmax loss function, some other loss function, or a composite of loss functions. The loss function can evaluate the degree to which trading data output vectors generated using the model 150 differ from the desired output (e.g., reference data output vectors) for corresponding training data images.

At block 218, the model training subsystem 116 can update parameters of the model 150 based on evaluation of the results of processing one or more training input images 400 using the model 150. The parameters may be updated so that if the same training data images are processed again, the output produced by the model 150 will be closer to the desired output represented by the reference data output vectors that correspond to the training data images. In some embodiments, the model training subsystem 116 may compute a gradient based on differences between the training data output vectors and the reference data output vectors. For example, gradient (e.g., a derivative) of the loss function can be computed. The gradient can be used to determine the direction in which individual parameters of the model 150 are to be adjusted in order to improve the model output (e.g., to produce output that is closer to the correct or desired output for a given input). The degree to which individual parameters are adjusted may be predetermined or dynamically determined (e.g., based on the gradient and/or a hyper parameter). For example, a hyper parameter such as a learning rate may specify or be used to determine the magnitude of the adjustment to be applied to individual parameters of the model 150.

With reference to an illustrative embodiment, the model training subsystem 116 can update some or all parameters of the model 150 (e.g., the weights of the model) using a gradient descent method with back propagation. In back propagation, a training error is determined using a loss function (e.g., as described above). The training error may be used to update the individual parameters of the model 150 in order to reduce the training error. For example, a gradient may be computed for the loss function to determine how the weights in the weight matrices are to be adjusted to reduce the error. The adjustments may be propagated back through the model 150 layer-by-layer.

At decision block 220, the model training subsystem 116 can in some embodiments determine whether one or more stopping criteria are met. For example, a stopping criterion can be based on the accuracy of the model 150 as determined using the loss function, the test set, or both. As another example, a stopping criterion can be based on the number of iterations (e.g., “epochs”) of training that have been performed, the elapsed training time, or the like. If the one or more stopping criteria are met, the routine 200 can proceed to block 222; otherwise, the routine 200 can return to block 214 or some other prior block of the routine 200.

At block 222, the model training subsystem 116 can store and/or distribute the trained model 150. For example, the trained model 150 can be distributed to one or more MRI evaluation systems for use in evaluating MRI images. Advantageously, the model 150 can produce output from MRI image input that is indicative of whether a lumbar ROI—typically detected using a SPECT/CT procedure—is present in the MRI image input without requiring the patient to also undergo a SPECT/CT procedure. Routine 200 may terminate at block 224.

### Example MRI-Based Evaluation Process

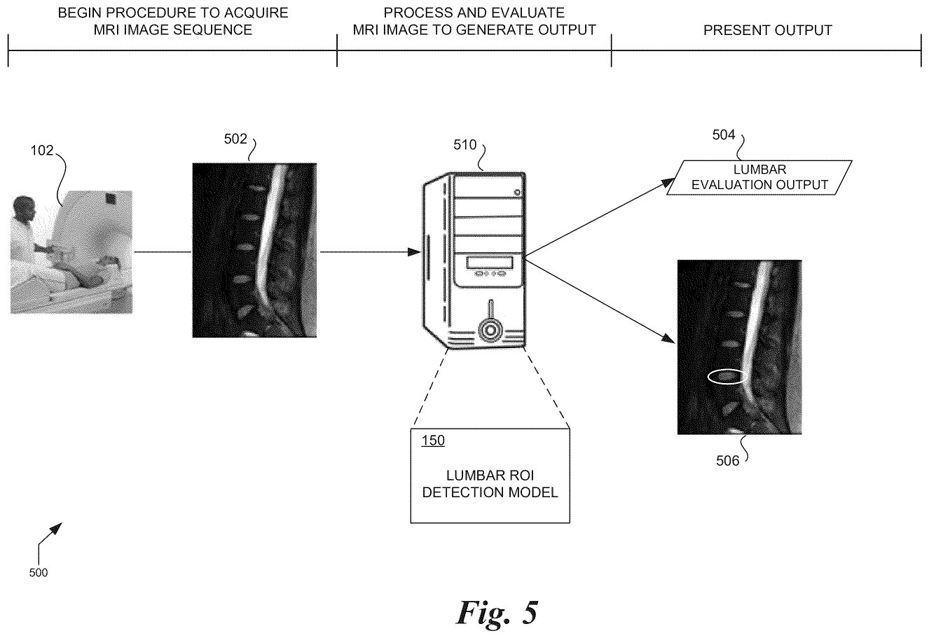

FIG. 5 illustrates data flows and interactions between devices of a lumbar region evaluation system 500 to perform an MRI imaging procedure and use a lumbar ROI detection model 150 to evaluate images generated during the procedure. The lumbar region evaluation system 500 may include an MRI machine 102 and an MRI evaluation system 510. It will be appreciated that the MRI evaluation system 510 may be integrated with the MRI machine 102 (e.g., in a single housing or physical location) or may be separate and remote from the MRI machine 102 (e.g., accessible via a wired or wireless network).

As shown, an MRI machine 102 may generate an MRI image 502 of a patient lumbar region. The MRI evaluation system 510 may use the MRI image 502 as MRI image input to an evaluation process that uses the model 150 to evaluate the MRI image 502. Lumbar region evaluation output generated using the model 150 and MRI image 502 may be generated in one or more forms. In some embodiments, lumbar region evaluation output 504 may include a classification score or determination regarding whether the MRI image 502 includes a lumbar ROI, the location of the lumbar ROI, the type of lumbar ROI, etc. In some embodiments, output of the evaluation process may presented in the form of a visual augmentation 506 applied to the MRI image to indicate the presence or location of a lumbar ROI (or absence thereof).

### Example Computing Devices

FIG. 6 illustrates an example training system computing device 600 that may be used in some embodiments to execute the processes and implement the features of the training system 100 described above. In some embodiments, the training system computing device 600 may include: one or more computer processors 602, such as physical central processing units (CPUs) or graphics processing units (GPUs); one or more network interfaces 604, such as a network interface cards (NICs); one or more computer readable medium drives 606, such as high density disks (HDDs), solid state drives (SSDs), flash drives, and/or other persistent non-transitory computer-readable media; and one or more computer readable memories 610, such as random access memory (RAM) and/or other volatile non-transitory computer-readable media. The network interface 604 can provide connectivity to one or more networks or computing devices. The computer processor 602 can receive information and instructions from other computing devices or services via the network interface 604. The network interface 604 can also store data directly to the computer-readable memory 610. The computer processor 602 can communicate to and from the computer-readable memory 610, execute instructions and process data in the computer-readable memory 610, etc.

The computer-readable memory 610 may include computer program instructions that the computer processor 602 executes in order to implement one or more embodiments. The computer-readable memory 610 can store an operating system 612 that provides computer program instructions for use by the computer processor 602 in the general administration and operation of the training system computing device 600. The computer-readable memory 610 can also include training data generation instructions 614 for generating training data to use in the training of machine learning models. The computer-readable memory 610 can also include machine learning model training instructions 616 for implementing training of machine learning models. The computer-readable memory 610 can further include computer program instructions and other data for implementing aspects of the present disclosure, such as the model 150 (or a portion thereof) that is being trained.

FIG. 6 also illustrates an example MRI evaluation system computing device 650 that may be used in some embodiments to execute the processes and implement the features of the MRI evaluation system 510 described above. MRI evaluation system computing device 650 may include components that are similar in some or all respects to components of the training system computing device 600 described above. For example, the MRI evaluation system computing device 650 may include: one or more computer processors 652, one or more network interfaces 654, one or more computer readable medium drives 656, and one or more computer-readable memories 660. The computer-readable memory 660 may include computer program instructions that the computer processor 652 executes in order to implement one or more embodiments. The computer-readable memory 660 can store an operating system 662 that provides computer program instructions for use by the computer processor 652 in the general administration and operation of the MRI evaluation system computing device 650. The computer-readable memory 660 can also include MRI evaluation instructions 664 for using a model 150 to analyze MRI images. The computer-readable memory 660 can further include computer program instructions and other data for implementing aspects of the present disclosure.

In some embodiments, as shown, the training system computing device 600 may provide a trained model 150 to the MRI evaluation system computing device 650. In some embodiments, the MRI evaluation system computing device 650 may also or alternatively provide operational data 670 to the training system computing device 600. For example, MRI evaluation system computing device 650 may use an initial machine learning model to evaluate MRI images. The MRI evaluation system computing device 650 may send operational data 670 including output from evaluation of MRI images, the MRI images themselves, labels or other data provided by HCPs or other users, or some combination thereof. Training system computing device 600 may use the operational data 670 to update the model, such as by performing re-training and generating an updated machine learning model. The updated machine learning model may then be provided to MRI evaluation system computing device 650, which may replace the initial machine learning model (prior version) with the updated machine learning model. This cycle may repeat on a predetermined or dynamically determined basis.

### Terminology

Depending on the embodiment, certain acts, events, or functions of any of the processes or algorithms described herein can be performed in a different sequence, can be added, merged, or left out altogether (e.g., not all described operations or events are necessary for the practice of the algorithm). Moreover, in certain embodiments, operations or events can be performed concurrently, e.g., through multi-threaded processing, interrupt processing, or multiple processors or processor cores or on other parallel architectures, rather than sequentially.

The various illustrative logical blocks, modules, routines, and algorithm steps described in connection with the embodiments disclosed herein can be implemented as electronic hardware, or combinations of electronic hardware and computer software. To clearly illustrate this interchangeability, various illustrative components, blocks, modules, and steps have been described above generally in terms of their functionality. Whether such functionality is implemented as hardware, or as software that runs on hardware, depends upon the particular application and design constraints imposed on the overall system. The described functionality can be implemented in varying ways for each particular application, but such implementation decisions should not be interpreted as causing a departure from the scope of the disclosure.

Moreover, the various illustrative logical blocks and modules described in connection with the embodiments disclosed herein can be implemented or performed by a machine, such as a processor device, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A processor device can be a microprocessor, but in the alternative, the processor device can be a controller, microcontroller, or state machine, combinations of the same, or the like. A processor device can include electrical circuitry configured to process computer-executable instructions. In another embodiment, a processor device includes an FPGA or other programmable device that performs logic operations without processing computer-executable instructions. A processor device can also be implemented as a combination of computing devices, e.g., a combination of a DSP and a microprocessor, a plurality of microprocessors, one or more microprocessors in conjunction with a DSP core, or any other such configuration. Although described herein primarily with respect to digital technology, a processor device may also include primarily analog components. For example, some or all of the algorithms described herein may be implemented in analog circuitry or mixed analog and digital circuitry. A computing environment can include any type of computer system, including, but not limited to, a computer system based on a microprocessor, a mainframe computer, a digital signal processor, a portable computing device, a device controller, or a computational engine within an appliance, to name a few.

The elements of a method, process, routine, or algorithm described in connection with the embodiments disclosed herein can be embodied directly in hardware, in a software module executed by a processor device, or in a combination of the two. A software module can reside in RAM memory, flash memory, ROM memory, EPROM memory, EEPROM memory, registers, hard disk, a removable disk, a CD-ROM, or any other form of a non-transitory computer-readable storage medium. An exemplary storage medium can be coupled to the processor device such that the processor device can read information from, and write information to, the storage medium. In the alternative, the storage medium can be integral to the processor device. The processor device and the storage medium can reside in an ASIC. The ASIC can reside in a user terminal. In the alternative, the processor device and the storage medium can reside as discrete components in a user terminal.

Conditional language used herein, such as, among others, “can,” “could,” “might,” “may,” “e.g.,” and the like, unless specifically stated otherwise, or otherwise understood within the context as used, is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without other input or prompting, whether these features, elements and/or steps are included or are to be performed in any particular embodiment. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list.

Disjunctive language such as the phrase “at least one of X, Y, Z,” unless specifically stated otherwise, is otherwise understood with the context as used in general to present that an item, term, etc., may be either X, Y, or Z, or any combination thereof (e.g., X, Y, and/or Z). Thus, such disjunctive language is not generally intended to, and should not, imply that certain embodiments require at least one of X, at least one of Y, or at least one of Z to each be present.

Unless otherwise explicitly stated, articles such as “a” or “an” should generally be interpreted to include one or more described items. Accordingly, phrases such as “a device configured to” are intended to include one or more recited devices. Such one or more recited devices can also be collectively configured to carry out the stated recitations. For example, “a processor configured to carry out recitations A, B and C” can include a first processor configured to carry out recitation A working in conjunction with a second processor configured to carry out recitations B and C.

While the above detailed description has shown, described, and pointed out novel features as applied to various embodiments, it can be understood that various omissions, substitutions, and changes in the form and details of the devices or algorithms illustrated can be made without departing from the spirit of the disclosure. As can be recognized, certain embodiments described herein can be embodied within a form that does not provide all of the features and benefits set forth herein, as some features can be used or practiced separately from others. The scope of certain embodiments disclosed herein is indicated by the appended claims rather than by the foregoing description. All changes which come within the meaning and range of equivalency of the claims are to be embraced within their scope.

The following is claimed:

1. A computer-implemented method for machine learning training for detection of lumbar regions of interest, the computer-implemented method comprising:
under control of a computer system comprising one or more processors configured to execute specific computer-executable instructions,
obtaining a plurality of Single Photon Emission Computed Tomography/Computed Tomography (SPECT/CT) images, wherein each SPECT/CT image of the plurality of SPECT/CT images is associated with a different patient of a plurality of patients, wherein a first subset of the plurality of SPECT/CT images represent patient lumbar regions with a lumbar region of interest, and wherein a second subset of the plurality of SPECT/CT images represent patient lumbar regions without a lumbar region of interest;
obtaining a plurality of magnetic resonance imaging (MRI) images, wherein each MRI image of the plurality of MRI images is associated with a corresponding SPECT/CT image of the plurality of SPECT/CT images;
labeling each MRI image of the plurality of MRI images based on the corresponding SPECT/CT image of the plurality of SPECT/CT images, wherein a label for a first MRI image indicates a presence of a lumbar region of interest in the first MRI image based on a presence of a lumbar region of interest in the corresponding SPECT/CT image; and
training a machine learning model using the plurality of MRI images, wherein the machine learning model is trained to generate model output regrading a presence of data representing a lumbar region of interest in model input.

2. The computer-implemented method of claim 1, wherein a lumbar region of interest in a SPECT/CT image corresponds to a lumbar region associated with larger uptake of radiotracer relative to a surrounding region of tissue.

3. The computer-implemented method of claim 1, wherein a lumbar region of interest in a SPECT/CT image corresponds to a patient lumbar region predicted to be experiencing facet joint arthropathy.

4. The computer-implemented method of claim 1, wherein a lumbar region of interest in a SPECT/CT image comprises a region of different color or brightness relative to a surrounding region of the SPECT/CT image.

5. The computer-implemented method of claim 1, further comprising distributing the machine learning model to an MRI evaluation system.

6. The computer-implemented method of claim 1, further comprising obtaining an initial version of the machine learning model, wherein the initial version of the machine learning model comprises a convolutional neural network.

7. The computer-implemented method of claim 6, wherein training the machine learning model comprises:
generating a training data output vector using the machine learning model and the first MRI image, wherein the training data output vector represents a classification of at least a portion of the first MRI image as one of negative or positive for a presence of data representing a lumbar region of interest;
computing a gradient based on a difference between the training data output vector and the label associated with the first MRI image; and
updating a parameter value of a plurality of parameter values of the machine learning model using the gradient.

8. The computer-implemented method of claim 7, further comprising determining the difference between the training data output vector and the label associated with the first MRI image using a loss function.

9. The computer-implemented method of claim 1, wherein labeling each MRI image of the plurality of MRI images comprises:
presenting a user interface displaying the first MRI image and the corresponding SPECT/CT image;
receiving, via the user interface, user input indicating a portion of at least one of the first MRI image or the corresponding SPECT/CT image associated with a lumbar region of interest; and
generating, based on the user input, the label for the first MRI image.

10. A lumbar disc evaluation system comprising:
a magnetic resonance imaging (MRI) machine configured to generate a first MRI image representing a lumbar region of a patient;
computer-readable memory storing a machine learning model trained to generate lumbar region evaluation output representing whether an MRI image input is associated with a lumbar region of interest; and
one or more processors in communication with the computer-readable memory, wherein the one or more processors are programmed by executable instructions to:
obtain the first MRI image generated by the MRI machine; and
generate a first lumbar region evaluation output based on evaluation of at least a portion of the first MRI image using the machine learning model.

11. The lumbar disc evaluation system of claim 10, further comprising a display, wherein the one or more processors are further programmed by the executable instructions to present at least the portion of the first MRI image on the display with a visual augmentation representing a presence of a lumbar region of interest based on first the lumbar region evaluation output.

12. The lumbar disc evaluation system of claim 10, further comprising a display, wherein the one or more processors are further programmed by the executable instructions to present the first lumbar region evaluation output on the display.

13. The lumbar disc evaluation system of claim 10, wherein a lumbar region of interest corresponds to a lumbar region in a SPECT/CT image of the lumbar region of the patient associated with larger uptake of radiotracer relative to a surrounding region of tissue.

14. The lumbar disc evaluation system of claim 10, wherein a lumbar region of interest corresponds to a lumbar region predicted to be experiencing facet joint arthropathy.

15. The lumbar disc evaluation system of claim 10, further comprising a network interface, wherein the one or more processors are further programmed by the executable instructions to:
send operational data to a training system remote from the lumbar disc evaluation system, wherein the operational data comprises at least one of the first lumbar region evaluation output or at least the portion of the first MRI image;
receive an updated machine learning model from the training system, wherein the updated machine learning model is trained based at least partly on the operational data; and
replace, in the computer-readable memory, the machine learning model with the updated machine learning model.
